(19) United States
(12) Patent Application Publication
(10) Pub. No.: US 2026/0191997 A1
(43) Pub. Date: Jul. 9, 2026

(54) CONJUGATES HAVING ANTIFOULING NANOPARTICLES AND METHODS OF USE

(71) Applicant: BOARD OF REGENTS, THE UNIVERSITY OF TEXAS SYSTEM, Austin, TX (US)

(72) Inventors: Jie ZHENG, Plano, TX (US); Mengxiao YU, Frisco, TX (US)

(21) Appl. No.: 18/704,210

(22) PCT Filed: Oct. 25, 2022
(86) PCT No.: PCT/US2022/047659

§ 371 Date: Apr. 24, 2024

Related U.S. Application Data
(60) Provisional application No. 63/271,638, filed on Oct. 25, 2021.

(51) Int. Cl. A61K 49/00; A61K 47/69; A61K 49/04; A61K 49/18; A61K 51/12; A61P 13/12

(52) U.S. Cl. CPC A61K 49/0002; A61K 47/6929; A61K 49/0034; A61K 49/0093; A61K 49/0438; A61K 49/049; A61K 49/1878; A61K 51/1244; A61P 13/12

(57) ABSTRACT

The present disclosure provides conjugates that comprise a small molecule attached to an antifouling nanoparticle. The present disclosure also provides methods of using such conjugates, such as diagnosis, tissue imaging, temporal monitoring, and treatment.

### RELATED APPLICATIONS

This application claims the benefit of U.S. Provisional Application No. 63/271,638, filed Oct. 25, 2021, the contents of which are incorporated herein by reference in its entirety.

### GOVERNMENT SUPPORT

This invention was made with government support under Grant Nos. R01 DK103363, R01 DK115986, R01 DK124881, and R44 CA268240 awarded by the National Institutes of Health. The government has certain rights in the invention.

### BACKGROUND

The kidneys are a major organ for rapid removal of endogenous wastes and exogenous drugs/toxins from the body. However, the exact elimination pathway for a solute strongly depends on its interactions with kidney compartments. Upon the interactions with kidney compartments, molecules can take two different pathways, glomerular filtration and renal tubular secretion, to be eliminated through the kidneys. For some molecules that have little interactions with kidney compartments and are smaller than 6 nm in hydrodynamic diameter (or lower than 40 kDa in molecular weight), they can be rapidly and passively eliminated through the glomerular filtration membrane. On the other hand, some other molecules can be actively excreted from peritubular capillaries into the lumen of the proximal tubules by binding the transporters on the basolateral side of proximal tubular cells, influx into the cells and efflux from the luminal side. The newly developed renal clearable nanofluorophores and organic dyes are mainly taking the glomerular filtration pathway and have been used for detecting kidney dysfunction or improving positive contrast of many cancers for fluorescence-guided surgery. However, none of them was reported to selectively target primary kidney cancers over normal kidney tissues and visualize the tumor margins with positive contrast (hyperfluorescence), which is highly demanded in fluorescence-guided partial nephrectomy to preserve kidney function and improve the quality of life of patients with kidney cancer. In addition, due to high blood perfusion of normal renal parenchyma, it remains challenging to selectively deliver therapeutic agents (such as agent for photothermal therapy, photodynamic therapy, chemotherapy, immunotherapy and radiation therapy) and medical imaging agents (such as agents for photoacoustic imaging, computed tomography, positron emission tomography, single-photon emission computerized tomography, and magnetic resonance imaging) into the kidney cancer cells at a higher concentration than nearby normal kidney tissues. Considering that over 90 percent of kidney cancer is renal cell carcinoma (RCC) originating from the renal tubular epithelial cell, the fundamental understanding of the interactions and transport of renal clearable dyes and nanoparticles with/in renal tubules is essential to designing new strategies for selectively targeting of RCC.

While proximal tubular secretion plays an important role in the rapid removal of endogenous substances and exogenous drugs or toxins, proximal tubules are also very vulnerable to endogenous cytokines and exogenous drugs or toxins, resulting in the impairment in tubular secretion function, kidney injury, and even kidney failure. Compared to glomerular filtration function that can be readily estimated with either endogenous serum creatinine or exogenous markers such as radiolabeling tracers or fluorescent inulin, tubular secretion function can only be quantified using few exogenous markers so far. In the clinics, exogenous functional markers such as para-aminohippurate (PAH) are intravenously infused into the patients. By analyzing their blood and urine concentrations with “off-line” colorimetric or chromatographic methods, clinicians can quantify remaining tubular secretion function and personalize treatment plans to minimize the potential side effects and nephrotoxicity. However, the “off-line” analysis is time-consuming and fails to address the urgent clinical needs in acute kidney injuries. In addition, the renal clearance of PAH is also affected by many other factors such as renal tubular blockage and cannot quantitatively report proximal tubular dysfunction and injury at high specificity. To address this challenge, radionuclides such as ⁹⁹mTc-mercaptoacetyltriglycine (MAG3) are being introduced for real-time monitoring of tubular secretion function; but potential radiation hazards and sophisticate clinical settings preclude them from being used in family clinics as well as in rural areas with limited medical resources. With the emergence of portable or wearable optoelectronics, it is highly desirable to develop exogenous nonradiative optical markers for remote assessment of proximal tubular secretion function and its impairment at the early stage at high specificity; so that prognostic planning and the early treatment can be timely made for patients in the remote areas with limited medical sources to prevent kidney failure.

### SUMMARY

In one aspect, the present disclosure provides conjugates comprising: a small molecule that binds an influx or efflux transporter; and an antifouling nanoparticle, wherein the small molecule is attached to the antifouling nanoparticle through a covalent bond or a metal-ligand bond, and when the small molecule is indocyanine green (ICG), the antifouling nanoparticle is not polyethylene glycol (PEG), and vice versa.

In one aspect, the present disclosure provides conjugates comprising ICG, PEG, and a secondary moiety, wherein ICG and the secondary moiety are each independently conjugated to PEG.

In one aspect, the present disclosure provides methods of diagnosing a disease or condition associated with abnormal expression of an influx or efflux transporter in a subject, comprising: (i) administering to the subject a conjugate described herein; (ii) determining a concentration of the conjugate in a biological sample obtained from the subject; (iii) comparing the concentration of the conjugate with a reference level; and (iv) determining that the subject has the disease or condition if the concentration of the conjugate is significantly greater or lower than the reference level.

In one aspect, the present disclosure provides methods of diagnosing a disease or condition associated with abnormal expression of an influx or efflux transporter in a subject, comprising: (i) administering to the subject a conjugate described herein; (ii) measuring an intensity of a signal from the conjugate in a tissue of the subject; (iii) comparing the intensity with a reference level; and (iv) determining that the subject has the disease or condition if the intensity is significantly greater or lower than the reference level.

In one aspect, the present disclosure provides methods of monitoring kidney secretion function of a subject, comprising: (i) administering to the subject a conjugate described herein; (ii) determining a first concentration of the conjugate in a first biological sample obtained from the subject at a first time point; (iii) determining a second concentration of the conjugate in a second biological sample obtained from the subject at a second time point, wherein the second time point is after the first time point; (iv) determining renal clearance kinetics based on the first concentration and the second concentration; and (v) optionally comparing the renal clearance kinetics with a reference level.

In one aspect, the present disclosure provides methods of monitoring kidney secretion function of a subject, comprising: (i) administering to the subject a conjugate described herein; (ii) measuring, at a first time point, a first intensity of a signal from the conjugate in a tissue of the subject; and (iii) measuring, at a second time point, a second intensity of a signal from the conjugate in the tissue of the subject.

In one aspect, the present disclosure provides methods of treating a disease or condition associated with abnormal expression of an influx or efflux transporter in a subject in need thereof, comprising administering to the subject a conjugate described herein.

In one aspect, the present disclosure provides methods of detecting a liver disease in a subject, comprising: (i) administering to the subject a conjugate described herein; (ii) determining a concentration of the conjugate in a biological sample obtained from the subject; (iii) comparing the concentration of the conjugate with a reference level; and (iv) determining that the subject has the liver disease when the concentration of the conjugate is significantly greater or lower than the reference level.

In one aspect, the present disclosure provides methods of measuring an expression level of an influx or efflux transporter in a subject, comprising: (i) administering to the subject a conjugate described herein; (ii) determining a concentration of the conjugate in a biological sample obtained from the subject; and (iii) determining the expression level of the influx or efflux transporter based on the concentration of the conjugate.

In one aspect, the present disclosure provides methods of measuring an expression level of an influx or efflux transporter in a subject, comprising: (i) administering to the subject a conjugate described herein; (ii) measuring an intensity of a signal from the conjugate in a tissue of the subject; and (iii) determining the expression level of the influx or efflux transporter based on the intensity.

### BRIEF DESCRIPTION OF THE DRAWINGS

FIG. 1 is a schematic diagram showing ICG-PEG45 labeled with Gd-DOTA (or ⁶⁴Cu-DOTA, ⁶⁷Cu-DOTA, ⁶⁸Ga-DOTA, ⁶⁷Ga-DOTA, ¹¹¹In-DOTA, ¹⁷⁷Lu-DOTA; DOTA=1,4,7,10-Tetraazacyclododecane-1,4,7,10-tetraacetic acid). They can be synthesized by three reactions: (a) Conjugation of maleimido-mono-amide-DOTA to a thiol terminated PEG45 (such as HS-PEG45-NH₂, HS-PEG45-ICG) through the thiol-maleimide reaction (HS-PEG45-NH₂═HS-PEG(2 kDa)-NH₂ in the scheme shown below); (b) Conjugation of ICG-NHS ester to an amine terminated PEG45 (such as DOTA-PEG45-NH₂, HS-PEG45-NH₂) through NHS ester-amine reaction; and (c) Coordination of DOTA to metal ions, such as Gd³⁺ and radioactive isotopes ⁶⁴Cu²⁺, ⁶⁷Cu²⁺, ⁶⁸Ga³⁺, ⁶⁷Ga³⁺, ¹¹¹In³⁺, ¹¹⁷Lu³⁺.

FIG. 2A is a noninvasive in vivo image of a mouse with orthotopic RCC xenograft on the left kidney at 24 h post intravenous injection of ICG-PE45-DOTA (200 μL, 40 μM). The left kidney of the mouse was implanted with papillary RCC cell line (ACHN) transfected with luciferase-expression vector, and right kidney was normal kept. At 10 min after intraperitoneal injection of luciferase substrate, strong bioluminescence signal was detected on the left kidney, indicating the growth of RCC. Bioluminescence image was overlapped with brightfield image to show the location of the bioluminescence signal on the left kidney.

FIG. 2B is a near-infrared fluorescence image of the same mouse suggests that ICG-PE45-DOTA can specifically accumulate in the left kidney with primary RCC but can be cleared from the normal right kidney (Ex/Em filters: 760/845 nm).

FIG. 3A is a bioluminescence image of the left and right kidneys that were cut in half longitudinally.

FIG. 3B is a photo of the kidneys shown in FIG. 3A.

FIG. 3C is a near-infrared fluorescence image of the kidneys shown in FIG. 3A. The fluorescent signal indicated that ICG-PE45-DOTA can specifically target the malignant kidney tissue (Ex/Em filters: 760/845 nm).

### DETAILED DESCRIPTION

### Definitions

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, may be branched, straight chain, or cyclic. Depending on the structure, an alkyl group can be a monoradical or a diradical (i.e., an alkylene group). The alkyl group could also be a “lower alkyl” having 1 to 6 carbon atoms.

As used herein, C₁-Cₓ includes, but is not limited to, C₁-C₂, C₁-C₃, C₁-C₄, C₁-C₅, C₁-C₆, C₂-C₃, C₂-C₄, C₂-C₅, C₂-C₆, C₃-C₄, C₃-C₅, C₃-C₆, C₄-C₅, C₄-C₆, and C₅-C₆.

The “alkyl” moiety may have 1 to 10 carbon atoms (whenever it appears herein, a numerical range such as “1 to 10” refers to each integer in the given range; e.g., “1 to 10 carbon atoms” means that the alkyl group may have 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups can be substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

As used herein, the term “non-cyclic alkyl” refers to an alkyl that is not cyclic (i.e., a straight or branched chain containing at least one carbon atom). Non-cyclic alkyls can be fully saturated or can contain non-cyclic alkenes and/or alkynes. Non-cyclic alkyls can be optionally substituted.

The term “alkenyl” refers to a type of alkyl group in which the first two atoms of the alkyl group form a double bond that is not part of an aromatic group. That is, an alkenyl group begins with the atoms —C(R)═C(R)—R, wherein R refers to the remaining portions of the alkenyl group, which may be the same or different. The alkenyl moiety may be branched, straight chain, or cyclic (in which case, it would also be known as a “cycloalkenyl” group). Depending on the structure, an alkenyl group can be a monoradical or a diradical (i.e., an alkenylene group). Alkenyl groups can be optionally substituted. Non-limiting examples of an alkenyl group include —CH═CH₂, —C(CH₃)═CH₂, —CH═CHCH₃, —C(CH₃)═CHCH₃. Alkenylene groups include, but are not limited to, —CH═CH—, —C(CH₃)═CH—, —CH═CHCH₂—, —CH═CHCH₂CH₂— and —C(CH₃)═CHCH₂—. Alkenyl groups could have 2 to 10 carbons. The alkenyl group could also be a “lower alkenyl” having 2 to 6 carbon atoms.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which may be the same or different. The “R” portion of the alkynyl moiety may be branched, straight chain, or cyclic. Depending on the structure, an alkynyl group can be a monoradical or a diradical (i.e., an alkynylene group). Alkynyl groups can be optionally substituted. Non-limiting examples of an alkynyl group include, but are not limited to, —C≡CH, —C≡CCH₃, —C≡CCH₂CH₃, —C≡C—, and —C≡CCH₂—. Alkynyl groups can have 2 to 10 carbons. The alkynyl group could also be a “lower alkynyl” having 2 to 6 carbon atoms.

An “alkoxy” group refers to a (alkyl)O— group, where alkyl is as defined herein.

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

An “alkenyloxy” group refers to a (alkenyl)O— group, where alkenyl is as defined herein.

The term “alkylamine” refers to the —N(alkyl)ₓH_{y} group, where x and y are selected from among x=1, y=1 and x=2, y=0. When x=2, the alkyl groups, taken together with the N atom to which they are attached, can optionally form a cyclic ring system.

“Alkylaminoalkyl” refers to an alkyl radical, as defined herein, substituted with an alkylamine, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, NY, 1999, which is incorporated herein by reference in its entirety.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, NY, 1999, which is incorporated herein by reference in its entirety.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g., aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e., a heteroatom). Heterocycle includes heteroaryl and heterocycloalkyl. Carbocycles and heterocycles can be optionally substituted.

The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

An “aryloxy” group refers to an (aryl)O— group, where aryl is as defined herein.

“Aralkyl” means an alkyl radical, as defined herein, substituted with an aryl group. Non-limiting aralkyl groups include, benzyl, phenethyl, and the like.

“Aralkenyl” means an alkenyl radical, as defined herein, substituted with an aryl group, as defined herein.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Illustrative examples of cycloalkyl groups include the following moieties:

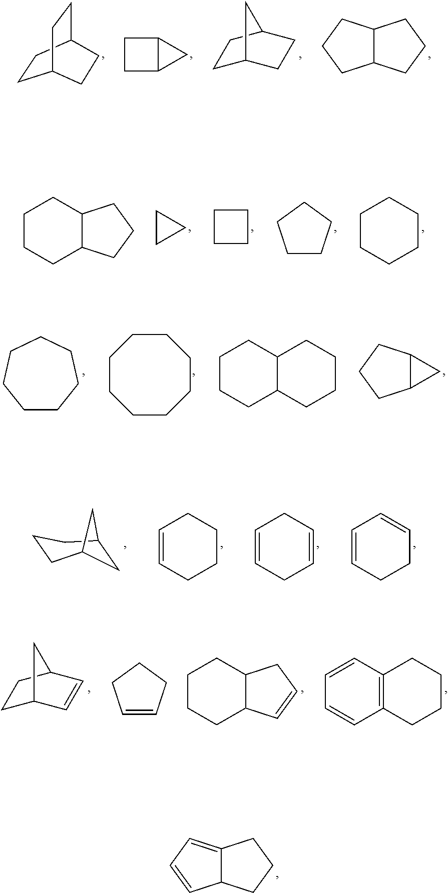

and the like. Depending on the structure, a cycloalkyl group can be a monoradical or a diradical (e.g., a cycloalkylene group). The cycloalkyl group could also be a “lower cycloalkyl” having 3 to 8 carbon atoms.

“Cycloalkylalkyl” means an alkyl radical, as defined herein, substituted with a cycloalkyl group. Non-limiting cycloalkylalkyl groups include cyclopropylmethyl, cyclobutylmethyl, cyclopentylmethyl, cyclohexylmethyl, and the like.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring can have additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms can be the same or different from one another. Heterocycles can be optionally substituted. Binding to a heterocycle can be at a heteroatom or via a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. Illustrative examples of heteroaryl groups include the following moieties:

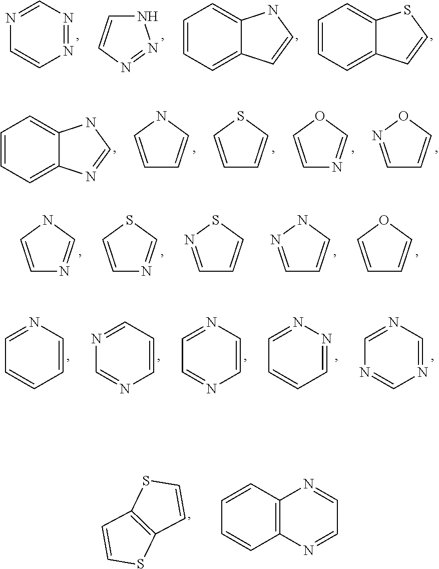

and the like. Depending on the structure, a heteroaryl group can be a monoradical or a diradical (i.e., a heteroarylene group).

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

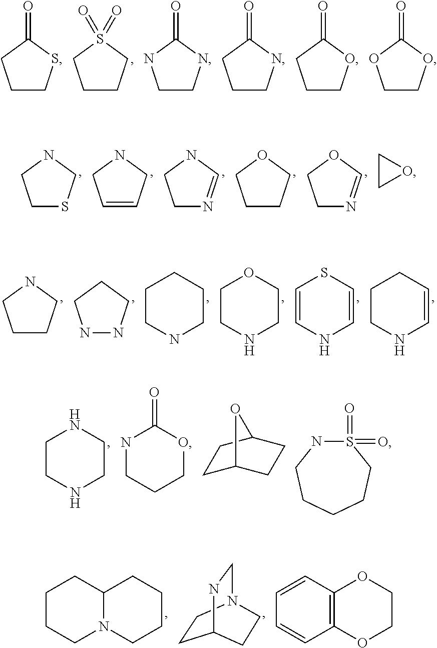

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

The term “halo” or, alternatively, “halogen” or “halide” means fluoro, chloro, bromo and iodo.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another.

The term “fluoroalkyl,” as used herein, refers to alkyl group in which at least one hydrogen is replaced with a fluorine atom. Examples of fluoroalkyl groups include, but are not limited to, —CF₃, —CH₂CF₃, —CF₂CF₃, —CH₂CH₂CF₃ and the like.

As used herein, the terms “heteroalkyl” “heteroalkenyl” and “heteroalkynyl” include optionally substituted alkyl, alkenyl and alkynyl radicals in which one or more skeletal chain atoms is a heteroatom, e.g., oxygen, nitrogen, sulfur, silicon, phosphorus or combinations thereof. The heteroatom(s) may be placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

A “thioalkoxy” or “alkylthio” group refers to a —S-alkyl group.

A “alkylthioalkyl” group refers to an alkyl group substituted with a —S-alkyl group.

As used herein, the term “O-carboxy” or “acyloxy” refers to a group of formula RC(═O)O—.

“Carboxy” means a —C(O)OH radical.

As used herein, the term “acetyl” refers to a group of formula —C(═O)CH₃.

“Acyl” refers to the group —C(O)R.

As used herein, the term “cyano” refers to a group of formula —CN.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, amino, including mono- and di-substituted amino groups, and the protected derivatives thereof. By way of example an optional substituents may be LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each Rₛ is independently selected from H, (substituted or unsubstituted C₁-C₄alkyl), (substituted or unsubstituted C₃-C₆cycloalkyl), heteroaryl, or heteroalkyl. The protecting groups that may form the protective derivatives of the above substituents are known to those of skill in the art and may be found in references such as Greene and Wuts, above.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “subject” as used herein, refer to either a human or a non-human animal. The term “subject” thus includes mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as pain, e.g., neuropathic pain. The skilled worker can readily determine the effective amount for a given situation by routine experimentation.

As used herein, the term “about” means a range of values that are similar to the stated reference value. In certain embodiments, the term “about” refers to a range of values that fall within 10 percent or less (e.g., 10%, 9%, 8%, 7%, 6%, 5%, 4%, 3%, 2%, or 1%) of the stated reference value.

As used herein, the term “significantly” means at least 5%, at least 10%, at least 15%, at least 20%, at least 25%, at least 30%, at least 35%, at least 40%, at least 45%, or at least 50%.

As used herein, the term “biochemically activatable agent” refers to an agent that can selectively react with biomolecules, enzymes, or ions.

As used herein, the term “small molecule” refers to a molecule having a molecular weight of less than 1500 Daltons. The small molecule can be an inorganic molecule, an organic molecule, a biomolecule, or an organometallic molecule.

### Conjugates

The present disclosure provides a variety of conjugates that are suitable for diagnostic and therapeutic applications. The conjugates have a one-hour or two-hour renal clearance efficiency in the range of 0.5 to 100 percent of injected dose (% ID), e.g., 0.5 to 90% ID, 0.5 to 90% ID, 5 to 100% ID, 5 to 90% ID, 10 to 100% ID, or 10 to 90% ID. In some embodiments, within 0.5-2 hours post intravenous injection, 5-100% of the injected conjugates are excreted through the kidney into the urine.

In one aspect, the present disclosure provides a conjugate comprising: a small molecule that binds an influx or efflux transporter; and an antifouling nanoparticle, wherein the small molecule is attached to the antifouling nanoparticle through a covalent bond or a metal-ligand bond, and when the small molecule is indocyanine green (ICG), the antifouling nanoparticle is not polyethylene glycol (PEG), and vice versa. In certain embodiments, the small molecule is capable of binding specifically one influx or efflux transporter. In certain embodiments, the small molecule is capable of binding multiple influx or efflux transporters on the proximal tubules.

In certain embodiments, the small molecule binds to an influx or efflux transporter with a dissociation constant of between about 0.1 nM and about 1 mM, between about 0.5 nM and about 0.5 mM, between about 1 nM and about 0.1 mM, or between about 1 nM and about 50 nM.

Influx transporters include, but are not limited to, organic anion transporter family (OATs, such as OAT1, OAT2, OAT3 and OAT4), organic anion transporting polypeptides (OATPs, such as OATP4A1 and OATP4C1), organic cation transporters family (OCTs, such as OCT2, OCT3), equilibrative nucleoside transporter 1 and 2 (ENT1 and ENT2), and organic solute transporter α and β (OSTα and OSTβ).

Efflux transporters include, but are not limited to, P-glycoprotein (P-gP; also termed multidrug resistance protein 1 (MDR1)), multidrug-resistant protein 2 and 4 (MRP2 and MRP4), organic cation transporters (OCTs, such as novel OCT (OCTN)1, OCTN2, multidrug and toxin exclusion (MATE) 1, and MATE kidney-specific 2), organic anion-transporting polypeptide family (OATPs), breast cancer resistance protein (BCRP), and organic anion transporter 4 (OAT4).

In certain embodiments, the small molecule that binds an influx or efflux transporter is a dye.

In certain embodiments, the dye is a near-infrared cyanine dye selected from ICG, IR-780, IR-783, MHI-148, and DZ-1. In certain embodiments, the dye is ICG.

In certain embodiments, the dye is a rhodamine dye selected from near-infrared dye Rhodamine 800, Rhodamine-123, Tetramethylrhodamine (TMR), Tetramethylrhodamine methyl ester (TMRM), and Rhodamine 6G.

In certain embodiments, the dye is a cationic carbocyanine dye selected from DiOC₆(3) (3,3′-dihexyloxacarbocyanine Iodide), DiOC₂(3) (3,3′-diethyloxacarbocyanine, iodide), and DisC₃(5) (3,3′-dipropylthiadicarbocyanine iodide).

In certain embodiments, the dye is fluorescein, a DNA stain (e.g., Hoechst 33342, DyeCycle Violet (DCV), Ethidium bromide, or SYBR Green), or bilirubin ditaurate.

In certain embodiments, the antifouling nanoparticle is an antifouling macromolecule. The antifouling macromolecule can be a biopolymer (e.g., a carbohydrate, a lipid, a protein, a peptide, or a nucleic acid). In certain embodiments, the antifouling macromolecule is a synthetic polymer.

In certain embodiments, the antifouling macromolecule is a hydrophilic polymer selected from polyether, polysaccharide, polyacrylamide, polyacrylate, polyamide, polypeptoids, β-peptoid, poly(β-peptoid)s, and polyalkyloxazoline. Exemplary hydrophilic polymers include ethylene glycol-based polymers, polyethylene glycol (PEG), poly(2-hydroxyethyl methacrylate) (pHEMA), poly(hydroxypropyl methacrylate) (pHPMA), dextran, and cellulose. In certain embodiments, the hydrophilic polymer is PEG.

In certain embodiments, the PEG has at least about 10, at least about 14, at least about 18, at least about 22, at least about 26, at least about 30, at least about 34, at least about 38, or at least about 42 repeating —CH₂CH₂O— units. In certain embodiments, the PEG has no more than about 1000, no more than about 950, no more than about 900, no more than about 850, no more than about 800, no more than about 750, no more than about 700, no more than about 650, no more than about 600, no more than about 550, no more than about 500, no more than about 450, or no more than about 400 repeating —CH₂CH₂O— units.

Combinations of the above-referenced ranges for the number of repeating —CH₂CH₂O— units are also possible (e.g., at least about 10 to no more than 900, at least about 22 to no more than about 700), inclusive of all values and ranges therebetween. For example, the number of repeating —CH₂CH₂O— units is about 22 to about 220, about 22 to about 44, about 43 to about 107, about 43 to about 90, about 43 to about 85, about 43 to about 80, about 43 to about 75, about 43 to about 70, about 43 to about 65, about 43 to about 60, or about 43 to about 55.

In certain embodiments, the PEG has about 42, 43, 44, 45, 46, 47, 48, 49, or 50 repeating —CH₂CH₂O— units. In certain embodiments, PEG has a molecular weight of about 2000 Da to about 6000 Da, e.g., about 2000 Da to about 4500 Da, about 2000 Da to about 4000 Da, about 2000 Da to about 3500 Da, or about 2000 Da to about 3000 Da.

In certain embodiments, the antifouling macromolecule is a zwitterionic polymer. Non-limiting examples of zwitterionic polymers include polybetaine or polyphosphorylcholine (with positive and negative charges in series on the same side chain), mixed cationic-anionic pairs (with positive and negative charges on two different side chains), and Bingdi cationic-anionic pairs (with positive and negative charges in parallel on the same side chain, such as cysteine).

In certain embodiments, the zwitterionic polymer is polycarboxybetaine, polysulfobetaine, or polyphosphorylcholine. Exemplary zwitterionic polymer in this category include poly(carboxybetaine methacrylate) (pCBMA), poly(sulfobetaine methacrylate) (pSBMA), or poly(2-methacryloyloxyethyl phosphorylcholine) (pMPC).

In certain embodiments, the zwitterionic polymer is poly(serine methacrylate) (pSerMA), poly(lysine methacrylamide) (pLysAA), poly(ornithine methacrylamide) (pOmAA), and polyampholyte mixed-charge copolymers composed of positively charged quaternary amine monomers or negatively charged monomers.

In certain embodiments, the zwitterionic polymer is a polyampholyte mixed-charge copolymer comprising positively charged quaternary amine monomers or negatively charged monomers. In certain embodiments, the positively charged quaternary amine monomer or negatively charged monomer is [2-(acryloyloxy)ethyl]trimethylammonium chloride or [2-(methacryloyloxy) ethyl]trimethylammonium chloride, 2-carboxy ethyl acrylate, or 3-sulfopropyl methacrylate potassium salt.

In certain embodiments, the antifouling macromolecule has an average molecular weight of at least about 500 Dalton, at least about 1,000 Dalton, at least about 1,500 Dalton, at least about 2,000 Dalton, at least about 2,500 Dalton, or at least about 3,000 Dalton. In certain embodiments, the antifouling macromolecule has an average molecular weight of at most about 3,000 Dalton, at most about 4,000 Dalton, at most about 5,000 Dalton, at most about 7,500 Dalton, at most about 10,000 Dalton, at most about 50,000 Dalton, or at most about 10,000 Dalton.

Combinations of the above-referenced ranges for the average molecular weight of the antifouling macromolecule are also contemplated. For example, in some embodiments, the antifouling macromolecule has an average molecular weight of about 500 to about 3,000 Dalton, about 500 to about 4,000 Dalton, about 1,000 to about 3,000 Dalton, about 1,000 to about 100,000 Dalton, about 2,000 to about 50,000 Dalton, about 3,000 to about 30,000 Dalton, or about 5,000 to about 10,000 Dalton.

In certain embodiments, the antifouling nanoparticle is an inorganic nanoparticle. For example, the inorganic nanoparticle comprises gold, silver, copper, platinum, palladium, silica, carbon, silicon, iron oxide, FeS, CdSe, CdS, CuS, or a combination thereof.

In certain embodiments, the antifouling nanoparticle has an average diameter of at least about 0.5 nm, at least about 0.75 nm, at least 1.0 nm, at least about 1.25 nm, at least about 1.5 nm, at least about 1.75 nm, or at least about 2.0 nm. In certain embodiments, the antifouling nanoparticle has an average diameter of at most about 15 nm, at most about 12 or at most about 8 nm.

Combinations of the above-referenced ranges for the average diameter of the antifouling nanoparticle are also contemplated. For example, in certain embodiments, the antifouling nanoparticle has an average diameter of about 0.5 nm to about 15 nm. In certain embodiments, the antifouling nanoparticle has an average diameter of about 0.5 nm to about 12 nm. In certain embodiments, the antifouling nanoparticle has an average diameter of about 0.5 nm to about 8 nm.

In certain embodiments, the conjugate can be further conjugated to an imaging agent, a biochemical activatable agent, or a therapeutic agent through a cleavable linker or a non-cleavable covalent bond. In certain embodiments, the small molecule is conjugated to the imaging agent, the biochemical activatable agent, or the therapeutic agent. In certain embodiments, the antifouling nanoparticle is conjugated to the imaging agent, the biochemical activatable agent, or the therapeutic agent. Non-limiting examples of the cleavable linkers include disulfide linker and enzyme cleavable linkers.

In certain embodiments, the imaging agent is a metal-ligand complex or an iodine-containing compound.

In certain embodiments, the imaging agent is a metal-ligand complex. Non-limiting examples of metal-ligand complexes include DOTA (1,4,7,10-Tetraazacyclododecane-1,4,7,10-tetraacetic acid), NOTA (1,4,7-triazacyclononane-1,4,7-triacetic acid), TETA (1,4,8,11-Tetraazacyclotetradecane-1,4,8, 11-tetraacetic acid), DTPA (diethylenetriaminepentaacetic acid), DO3A-EOB (1,4,7,10-tetraazacyclododecane-1,4,7-trisacetic Acid (DO3A)-Ethoxybenzyl (EOB)), DOTP (1,4,7,10-tetraazacyclododecane-1,4,7,10-tetra(methylene phosphonic) acid), Cyclen (1,4,7,10-Tetraazacyclododecane), Cyclam (1,4,8,11-Tetraazacyclotetradecane), CB-TE2A (1,4,8,11-tetraazabicyclo[6.6.2]hexadecane-4,11-diacetic acid), DOTMA ((1R, 4R, 7R, 10R)-α′α″α′″-tetramethyl-1,4,7,10-tetraazacyclododecane-1,4,7,10-tetraacetic acid) tetrasodium salt), or DFO (deferoxamine).

In certain embodiments, the metal in the metal-ligand complex is Gd, ⁶⁴Cu, ⁶⁷Cu, ⁶⁸Ga, ⁶⁷Ga, ¹¹¹In, ⁸⁹Zr, or ¹⁷⁷Lu.

In certain embodiments, the imaging agent is an iodine-containing compound. Non-limiting examples of iodine-containing compounds include an iodobenzene, a diiodobenzene, a triiodobenzene, and derivatives thereof (e.g., diatrizoate, metrizoate, iohexol, iopamidol, iopromide, ioxilan, iodixanol, iobitridol, ioversol, iothalamate, and ioxaglate).

In certain embodiments, the imaging agent is a gadolinium (Gd) complex (e.g., gadoversetamide, gadopentetate dimeglumine, gadoterate meglumine, gadoxetate disodium, gadoteridol, or gadobutrol), a dysprosium (Dy) complex (e.g., Dy-EOB-DTPA [(4S)-4-(4-ethoxybenzyl)-3,6,9-tris-(carboxylatomethyl)-3,6,9-triazaundecanedioic acid, dysprosium complex, or disodium salt), or a tantalum oxide-based contrast agent.

In certain embodiments, the imaging agent is for positron emission tomography (PET) and is a compound labeled by a radioisotope (e.g., ¹¹C, ¹⁸F, ⁶⁴Cu, ⁶⁸Ga, or ⁸⁹Zr).

In certain embodiments, the imaging agent is for single-photon emission computerized tomography (SPECT) and is a compound labeled by a radioisotope (e.g., ^{99m}Tc, ¹²³I, ¹²⁵I, ¹³¹I, ⁶⁷Ga, or ¹¹¹In).

In certain embodiments, the imaging agent is a gadolinium (Gd)-based MRI contrast agent, manganese (Mn)-based MRI contrast agent, or an iron oxide nanoparticle for magnetic resonance imaging (MRI). Non-limiting examples of gadolinium (Gd)-based MRI contrast agents include gadoversetamide, gadopentetate dimeglumine, gadoterate meglumine, gadoxetate disodium, gadoteridol, and gadobutrol. Non-limiting examples of manganese (Mn)-based MRI contrast agents include semistable chelate manganese dipyridoxyl diphosphate (MnDPDP), Mn chelated with ethylenediaminetetraacetic acid (Mn-EDTA), and Mn chelated with diethylene triamine pentaacetic acid (Mn-DTPA).

In certain embodiments, the therapeutic agent comprises an antioxidant used for preventing or treating oxidative stress-related diseases (e.g., N-acetylcysteine, alfa-lipoic acid, or dihydrolipoic acid).

In certain embodiments, the therapeutic agent comprises a small molecule drug for chemotherapy (e.g., cisplatin, carboplatin, doxorubicin, epirubicin, paclitaxel, docetaxel, methotrexate, capecitabine, sorafenib, sirolimus, temsirolimus, or everolimus).

In certain embodiments, the therapeutic agent comprises a compound containing a therapeutic radioisotope (e.g, ¹⁷Lu, ⁶⁷Cu, or ¹⁹⁸Au).

In certain embodiments, the therapeutic agent comprises a peptide, an antibody or a fragment thereof, an immunoagent, a nucleic acid (e.g., a RNA, a mRNA, or a DNA).

In certain embodiments, the conjugate fluoresces in the range of 500 nm to 850 nm. In certain embodiments, the conjugate fluoresces in the range of 1000 nm to 1700 nm.

In another aspect, the present disclosure provides a conjugate comprising ICG, PEG, and a secondary moiety, wherein ICG and the secondary moiety are each independently conjugated to PEG. In certain embodiments, the secondary moiety comprises 1,4,7,10-tetraazacyclododecane-1,4,7,10-tetraacetic acid (DOTA). In certain embodiments, the secondary moiety does not comprise DOTA.

In certain embodiments, the secondary moiety is an imaging agent as described herein. In certain embodiments, the secondary moiety is a therapeutic agent as described herein.

In certain embodiments, the ICG-PEG-secondary moiety conjugate is of formula I:

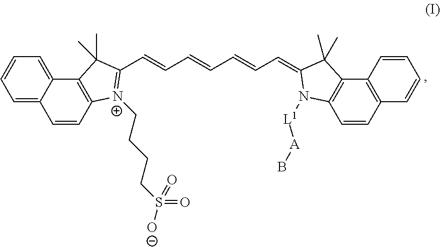

or a pharmaceutically acceptable salt thereof, wherein:
L¹ is independently optionally substituted alkylene, haloalkylene, alkenylene or alkynylene;
A is independently —C(O)NH(CH₂CH₂O)ₙ—, —C(O)O(CH₂CH₂O)ₙ—, —C(O)S(CH₂CH₂O)ₙ—, —NHC(O)CH₂O(CH₂CH₂O)ₙ—, —OC(O)CH₂O(CH₂CH₂O)ₙ—, or —SC(O)CH₂O(CH₂CH₂O)ₙ—, wherein the (CH₂CH₂O)— end is connected to B;
n is an integer selected from about 10 to about 1000; and
B is independently H, or optionally substituted alkyl.

In certain embodiments, L¹ is unsubstituted C₁₋₆ alkylene or C₁₋₆ haloalkylene.

In certain embodiments, B is H or unsubstituted C₁₋₆ alkyl.

In certain embodiments, B is C₁₋₆ alkyl substituted with one or more —OH, —NH₂, —SH, or —COOH.

In certain embodiments, B is —CH₂CH₂OH, —CH₂CH₂NH₂, —CH₂CH₂SH, —CH₂CH₂C(O)OH, or —CH₂C(O)OH.

Optionally, B is connected to a secondary moiety.

In certain embodiments, n is at least about 10, at least about 14, at least about 18, at least about 22, at least about 26, at least about 30, at least about 34, at least about 38, or at least about 42. In certain embodiments, n is no more than about 1000, no more than about 950, no more than about 900, no more than about 850, no more than about 800, no more than about 750, no more than about 700, no more than about 650, no more than about 600, no more than about 550, no more than about 500, no more than about 450, or no more than about 400.

Combinations of the above-referenced ranges for n are also possible (e.g., at least about 10 to no more than 900, at least about 22 to no more than about 700), inclusive of all values and ranges therebetween.

In certain embodiments, n is an integer selected from about 22 to about 220. In certain embodiments, n is an integer selected from about 22 to about 44. In certain embodiments, n is an integer selected from about 43 to about 107, e.g., from about 43 to about 90, from about 43 to about 85, from about 43 to about 80, from about 43 to about 75, from about 43 to about 70, from about 43 to about 65, from about 43 to about 60, or from about 43 to about 55.

In certain embodiments, n is 42, 43, 44, 45, 46, 47, 48, 49, or 50. In certain embodiments, n is 22. In certain embodiments, n is 220.

In certain embodiments, PEG has a molecular weight of about 2000 Da to about 6000 Da, e.g., about 2000 Da to about 4500 Da, about 2000 Da to about 4000 Da, about 2000 Da to about 3500 Da, or about 2000 Da to about 3000 Da.

In certain embodiments, the ICG-PEG-secondary moiety conjugate is in the form of nanoparticles. In certain embodiments, the nanoparticles have an average diameter of about 0.5 nm to about 12 nm, e.g., about 0.5 nm to about 10 nm, about 0.5 nm to about 8 nm, about 0.5 nm to about 6 nm, about 1 nm to about 12 nm, about 1 nm to about 10 nm, about 1 nm to about 8 nm, or about 1 nm to about 6 nm.

In another aspect, the present disclosure provides a pharmaceutical composition comprising the conjugate of the present disclosure and a pharmaceutically acceptable carrier.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); intravenously; subcutaneously; transdermally (for example as a patch applied to the skin); and topically (for example, as a cream, ointment or spray applied to the skin). The conjugate may also be formulated for inhalation. In certain embodiments, the conjugate may be simply dissolved or suspended in sterile water.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Formulations suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a conjugate of the present disclosure as an active ingredient. Compositions or conjugates may also be administered as a bolus, electuary or paste.

### Methods of Using Conjugates

Each of the conjugates described herein can be used in a variety of applications, including, but not limited to, diagnostic and therapeutic applications.

In one aspect, the conjugates described herein can be used to measure an expression level of an influx or efflux transporter in a subject. In some embodiments, the conjugate can be used to identify the differences in expression of influx or efflux transporters among healthy people but with differences in gender, age, etc. This can provide useful information for personalized medicine.

Accordingly, the present disclosure provides a method of measuring an expression level of an influx or efflux transporter in a subject, comprising: (a) administering to the subject a conjugate described herein; (b) determining a concentration of the conjugate in a biological sample obtained from the subject; and (c) determining the expression level of the influx or efflux transporter based on the concentration of the conjugate.

The present disclosure also provides a method of measuring an expression level of an influx or efflux transporter in a subject, comprising: (a) administering to the subject a conjugate described herein; (b) measuring an intensity of a signal from the conjugate in a tissue of the subject; and (c) determining the expression level of the influx or efflux transporter based on the intensity.

In certain embodiments, the expression level is the absolute expression level. In certain embodiments, the expression level is the relative expression level. For example, the expression level can be relative to that in a population with different gender and/or age.

In one aspect, the conjugate described herein can be used as a marker for diagnostic applications, including but not limited to, monitoring influx transporter activities, monitoring efflux transporter activities, monitoring kidney secretion function, monitoring liver function, and diagnosing or detecting a disease or condition associated with abnormal expression of an influx or efflux transporter. In certain embodiments, the conjugate described herein can be used as an exogenous marker, e.g., for blood or urine samples.

Accordingly, the present disclosure provides a method of diagnosing a disease or condition associated with abnormal expression of an influx or efflux transporter in a subject, comprising: (a) administering to the subject a conjugate described herein; (b) determining a concentration of the conjugate in a biological sample obtained from the subject; (c) comparing the concentration of the conjugate with a first reference level; and (d) determining that the subject has the disease or condition if the concentration of the conjugate is significantly greater or lower than the first reference level.

Abnormal expression of an influx transporter can mean either upregulation or downregulation of the influx transporter as compared to the expression level in a normal tissue. Abnormal expression of an efflux transporter can mean either upregulation or downregulation of the efflux transporter as compared to the expression level in a normal tissue.

In certain embodiments, the first reference level is the concentration of the conjugate in a corresponding biological sample obtained from a healthy subject. In certain embodiments, the healthy subject is of the same gender and/or similar age as the subject.

The present disclosure also provides a method of monitoring kidney secretion function of a subject, comprising: (a) administering to the subject a conjugate described herein; (b) determining a first concentration of the conjugate in a first biological sample obtained from the subject at a first time point; (c) determining a second concentration of the conjugate in a second biological sample obtained from the subject at a second time point, wherein the second time point is after the first time point; (d) determining renal clearance kinetics based on the first concentration and the second concentration; and (e) optionally comparing the renal clearance kinetics with a second reference level.

In certain embodiments, the first time point can be at least about 1 minute, at least about 30 minutes, at least about one hour, at least about 90 minutes, or at least about two hours after the conjugate is administered. In certain embodiments, the second time point can be at least about 5 minutes, at least about 30 minutes, at least about one hour, at least about 90 minutes, at least about two hours, or more after the first time point. To determine the renal clearance kinetics, more than two time points (e.g., three, four, or five time points) can be utilized.

In certain embodiments, the method further comprises determining that the subject has abnormal kidney secretion function if the renal clearance kinetics is significantly greater or less than the second reference level.

In certain embodiments, the second reference level is the renal clearance kinetics of a healthy subject. In certain embodiments, the healthy subject is of the same gender and/or similar age as the subject.

In certain embodiments, the biological sample can be blood, plasma, serum, urine, a tissue biopsy, a cell, a plurality of cells, fecal matter, or saliva. In certain embodiments, the biological sample is a blood sample. In certain embodiments, the biological sample is a urine sample.

Without wishing to be bound by theory, as the conjugate with molecular weight of the antifouling nanoparticle smaller than 2 kDa can be eliminated through both the liver and kidneys, liver injury will slow down its elimination through the liver pathway, but increase its renal clearance. Accordingly, certain conjugates can be used to detect liver diseases. The present disclosure provides a method of detecting a liver disease in a subject, comprising: (a) administering to the subject a conjugate described herein, wherein the antifouling nanoparticle has a molecular weight of less than 2 kDa; (b) determining a concentration of the conjugate in a urine sample obtained from the subject; (c) comparing the concentration of the conjugate with a third reference level; and (d) determining that the subject has the liver disease when the concentration of the conjugate is significantly less than the third reference level.

In certain embodiment, the conjugate with the antifouling nanoparticle molecular weight between about 100 Da and about 2 kDa can be eliminated through both the liver and kidneys. Accordingly, in certain embodiments, the antifouling nanoparticle in the conjugate used for detecting liver diseases has a molecular weight of at least about 100 Da to less than about 2 kDa, e.g., between about 100 Da and about 1800 Da, between about 100 Da and about 1600 Da, between about 100 Da and about 1400 Da, between about 100 Da and about 1200 Da, between about 100 Da and about 1000 Da, between about 100 Da and about 800 Da, between about 100 Da and about 600 Da, between about 200 Da and about 1800 Da, between about 200 Da and about 1600 Da, between about 200 Da and about 1400 Da, between about 200 Da and about 1200 Da, between about 200 Da and about 1000 Da, between about 200 Da and about 800 Da, or between about 200 Da and about 600 Da.

In certain embodiments, the third reference level is the concentration of the conjugate in a urine sample obtained from a healthy subject. In certain embodiments, the healthy subject is of the same gender and/or similar age as the subject.

In another aspect, the conjugate described herein can be used as an imaging agent configured to produce a signal with measurable intensity. By measuring the intensity of a signal from the conjugate after it is administered to a subject, it permits a technician/physician to identify diseased tissues, monitor influx transporter activities, monitor efflux transporter activities, or detect a disease or condition associated with abnormal expression of an influx or efflux transporter.

In certain embodiments, measuring an intensity of a signal from the conjugate in a tissue merely measures the intensity without spatial information.

In certain embodiments, measuring an intensity of a signal from the conjugate in a tissue comprises imaging the tissue so as to produce both the intensity and spatial information of the conjugate in the tissue. In certain embodiments, the conjugate can be used as a positive image contrast agent.

Accordingly, the present disclosure provides a method of monitoring kidney secretion function of a subject, comprising: (a) administering to the subject a conjugate described herein; (b) measuring, at a first time point, a first intensity of a signal from the conjugate in a tissue of the subject; and (c) measuring, at a second time point, a second intensity of a signal from the conjugate in the tissue of the subject. This method can thus permit temporal monitoring of kidney secretion function.

In certain embodiments, the method of monitoring kidney secretion function further comprises comparing the second intensity with the first intensity and determining that the kidney secretion function is abnormal or deteriorating if the second intensity is significantly higher or lower than the first intensity.

In certain embodiments, the method of monitoring kidney secretion function further comprises determining renal clearance kinetics based on the first intensity and the second intensity; and (e) optionally comparing the renal clearance kinetics with the second reference level, as discussed above.

In certain embodiments, the first time point can be at least about 1 minute, at least about 30 minutes, at least about one hour, at least about 90 minutes, or at least about two hours after the conjugate is administered. In certain embodiments, the second time point can be at least about 5 minutes, at least about 30 minutes, at least about one hour, at least about 90 minutes, at least about two hours, or more after the first time point. The method of monitoring kidney secretion function can utilize more than two time points (e.g., three, four, or five time points) to determine the intensity kinetics. Each time point can be separated from its prior time point by at least about 5 minutes, at least about 30 minutes, at least about one hour, at least about 90 minutes, at least about two hours, or more.

In certain embodiments, the intensity is measured as frequently as needed, e.g., every three hours, every 2.5 hours, every two hours, every 1.5 hours, every hour, every 30 minutes, every 20 minutes, every 10 minutes, or every 5 minutes.

In certain embodiments, the measurements are performed for as long as needed, e.g., in a few hours to a few weeks, e.g., about 6 hours, about 12 hours, about 18 hours, about 24 hours, about two days, about four days, about eight days, or about 16 days.

In certain embodiments, the method of monitoring kidney secretion function further comprises determining that the subject has abnormal kidney secretion function if the renal clearance kinetics is significantly greater or less than the second reference level. Abnormal kidney secretion function can then be used to diagnose a disease or condition associated with abnormal expression of an influx or efflux transporter.

The present disclosure also provides a method of diagnosing a disease or condition associated with abnormal expression of an influx or efflux transporter in a subject, comprising: (a) administering to the subject a conjugate described herein; (b) measuring an intensity of a signal from the conjugate in a tissue of the subject; (c) comparing the intensity with a fourth reference level; and (d) determining that the subject has the disease or condition if the intensity is significantly greater or lower than the fourth reference level.

The conjugate can provide positive image contrast of a diseased renal tubular tissue with influx and/or efflux transporters upregulated or downregulated as compared to surrounding normal tissues. The conjugate can also provide positive image contrast of diseases in other organs and tissues than the kidney. For instance, the conjugate can selectively accumulate in kidney cancer metastases in the brain, bone, lungs, etc. The rapid elimination from the background and normal tissues further enhances imaging contrast. In addition, the conjugate also can target other diseases related to the upregulation or downregulation of transporters that the conjugate binds to. For instance, MCF-7 triple negative breast tumor can also be selectively targeted with the conjugate because OATP1A2 is overexpressed, so that selective accumulation of the conjugate in MCF-7 tumor is observed in the tumor-bearing mice.

In certain embodiments, measuring the intensity comprises imaging the tissue, which provides a contrast index of at least about 1.5. In certain embodiments, the fourth reference level is the intensity of a signal from the conjugate in a corresponding normal tissue of the subject or a healthy subject. In certain embodiments, the healthy subject is of the same gender and/or similar age as the subject.

In certain embodiments, the intensity is significantly greater or lower than the fourth reference level due to either upregulated or downregulated transporters compared to nearby normal tissues or normal status.

Measuring the intensity can utilize fluorescence imaging, photoacoustic imaging, computed tomography (CT), positron emission tomography (PET), single-photon emission computerized tomography (SPECT), magnetic resonance imaging (MRI), or photothermal imaging. The tissue can be blood, a body part or a portion thereof, an organ or a portion thereof, or a diseased tissue such as a tumor. For example, the organ can be kidney or bladder. Measuring the intensity can be performed either invasively (i.e., on a biological sample obtained from a subject) or noninvasively. With respect to noninvasive measurements, a variety of devices can be used to measure the intensity of the signal from the conjugate. For example, a transdermal optical device (e.g., a finger clip oximeter) can be used. In certain embodiments, a portable transdermal optical device is used to measure the intensity of the signal from the conjugate.

Depending on the method used to measure the signal, the signal can be an optical signal (e.g., fluorescence), an ultrasonic signal, a radioactive signal (e.g., X-ray signal or gamma-ray signal), or a radio wave.

Measuring the intensity can provide temporal and/or spatial information of the tissue.

In yet another aspect, the present disclosure provides a method of treating a disease or condition associated with abnormal expression of an influx or efflux transporter in a subject in need thereof, comprising administering to the subject a conjugate described herein.

Depending on the composition of the conjugate, the treatment methods can vary. For example, some of the small molecules that are capable of binding an efflux or influx transporter can be photothermal or photodynamic agent, so after the conjugate is administered, electromagnetic radiation can be applied to the subject to treat the disease or condition through photothermal or photodynamic therapy. In certain embodiments where the small molecules that are capable of binding an efflux or influx transporter are therapeutic radioisotopes, after the conjugate is administered, radiation therapy can be applied to the subject to treat the disease or condition.

The conjugate can also serve as a drug-delivery vector for disease treatment. The selective binding of the conjugate to the influx or efflux transporter permits it to be used as drug delivery vectors that can direct the medical agents to selectively accumulate inside the diseased cells. The drugs can be loaded on the antifouling nanoparticles through non-covalent interactions or covalent interactions. The loaded drugs can be released from the antifouling nanoparticles by chemical or optical stimulation after entering the diseased cells. The medical agents loaded onto the engineered nanoparticles include, but are not limited to, antioxidants, chemodrugs, therapeutic radioisotopes, inhibitors, peptides, antibodies or fragments thereof, immunoagents, RNAi, mRNAs, DNAs, etc. The diseases include, but are not limited to, acute kidney injury, kidney cancer, cysts, breast cancers, and metastatic cancer.

In certain embodiments of any one of the above aspects, influx transporters include, but are not limited to, organic anion transporter family (OATs, such as OAT1, OAT2, OAT3 and OAT4), organic anion transporting polypeptides (OATPs, such as OATP4A1 and OATP4C1), organic cation transporters family (OCTs, such as OCT2, OCT3), equilibrative nucleoside transporter 1 and 2 (ENT1 and ENT2), and organic solute transporter α and β (OSTα and OSTβ).

In certain embodiments of any one of the above aspects, efflux transporters include, but are not limited to, P-glycoprotein (P-gP; also termed multidrug resistance protein 1 (MDR1)), multidrug-resistant protein 2 and 4 (MRP2 and MRP4), organic cation transporters (OCTs, such as novel OCT (OCTN)1, OCTN2, multidrug and toxin exclusion (MATE) 1, and MATE kidney-specific 2), organic anion-transporting polypeptide family (OATPs), breast cancer resistance protein (BCRP), and organic anion transporter 4 (OAT4).

Other influx/efflux transporters include transporters of peptides, PDZ Domains, Type 1 Sodium/Phosphate Co-Transport (NPT1), URAT1, BSP/Bilirubin binding protein (BBBP).

In certain embodiments of any one of the above aspects, the subject has upregulated or downregulated expression of P-glycoprotein (P-gp), multidrug-resistant protein 2 (MRP2), MRP4, an organic cation transporter (OCT), an organic anion transporter (OAT), an organic anion-transporting polypeptide (OATP), breast cancer resistance protein (BCRP), or organic anion transporter 4 (OAT4), equilibrative nucleoside transporter 1 (ENT1), ENT2, organic solute transporter α (OSTα), or OSTβ.

In certain embodiments of any one of the above aspects, the disease or condition associated with abnormal expression of an influx or efflux transporter is renal tubular secretion dysfunction or renal tubular injury.

In certain embodiments of any one of the above aspects, the renal tubular secretion dysfunction or renal tubular injury is proximal renal tubular secretion dysfunction or proximal renal tubular injury.

In certain embodiments of any one of the above aspects, the renal tubular secretion dysfunction or renal tubular injury is associated with a kidney disease or condition selected from acute kidney injury, chronic kidney injury, kidney cancer, lupus nephritis, diabetes-induced kidney injury, polycystic kidney disease, sepsis, kidney inflammation, kidney transplant rejection, and kidney dysfunction or kidney injury caused by diseases in other tissues and organs such as cancer and liver diseases.

In certain embodiments of any one of the above aspects, the disease or condition associated with abnormal expression of an influx or efflux transporter is kidney cancer, breast cancer, liver cancer, ovarian cancer, bladder cancer, prostate cancer, lung cancer, pancreatic cancer, bone cancer, or colon cancer, or their metastases in other organs or normal tissues.

In certain embodiments, the kidney cancer is renal cell carcinoma or renal oncocytoma, or their metastases in other organs or normal tissues.

In certain embodiments, the kidney cancer is renal cell carcinoma. In certain embodiments, the renal cell carcinoma is clear call renal cell carcinoma (ccRCC), or papillary RCC (pRCC).

In certain embodiments, the breast cancer is triple negative breast cancer, or their metastases in other organs or normal tissues. In certain embodiments, the triple negative breast cancer is 4T1 or MCF-7 triple negative breast.

In certain embodiments of any one of the above aspects, the conjugate is administered intravenously, intraperitoneally, subcutaneously, or intraarterially. In certain embodiments of any one of the above aspects, the conjugate is administered intravenously.

In certain embodiments of any one of the above aspects, the kidney can be native to the subject. In certain embodiments of any one of the above aspects, the kidney is a donor kidney for transplant and the conjugate is administered to the blood vessel that is connected to the donor kidney for transplant.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1. ICG-PEG45 Labeled with DOTA

PEG samples with average molecular weight of 1100 Da, 2100 Da, 3500 Da, 5000 Da and 10100 Da were purchased from Sigma-Aldrich (USA). ICG-NHS and IRDye800CW-NHS were purchased from Intrace Medical (Switzerland) and LI-COR, respectively. Absorption spectra were measured by a Virian 50 Bio UV-vis spectrophotometer. Fluorescence spectra were acquired by a PTI QuantaMaster™ 30 Fluorescence Spectrophotometer (Birmingham, NJ). In vivo fluorescence images were recorded using a Carestream In-vivo FX Pro imaging system. Optical images of cultured cells and tissue slides were obtained with an Olympus IX-71 inverted fluorescence microscope coupled with Photon Max 512 CCD camera (Princeton Instruments). Agarose gel electrophoresis was carried out by a Bio-Rad Mini-Sub Cell GT system. Animal studies were performed according to the guidelines of the University of Texas System Institutional Animal Care and Use Committee. BALB/c mice (BALB/cAnNCr, strain code 047) of 6-8 weeks old, weighing 20-25 g, were purchased from Envigo. Nude mice (Athymic NCr-nu/nu, strain code 069) of 6-8 weeks old, weighing 20-25 g, were also purchased from Envigo. All of these mice were randomly allocated and housed under standard environmental conditions (23±1° C., 50±5% humidity and a 12/12 h light/dark cycle) with free access to water and standard laboratory food.

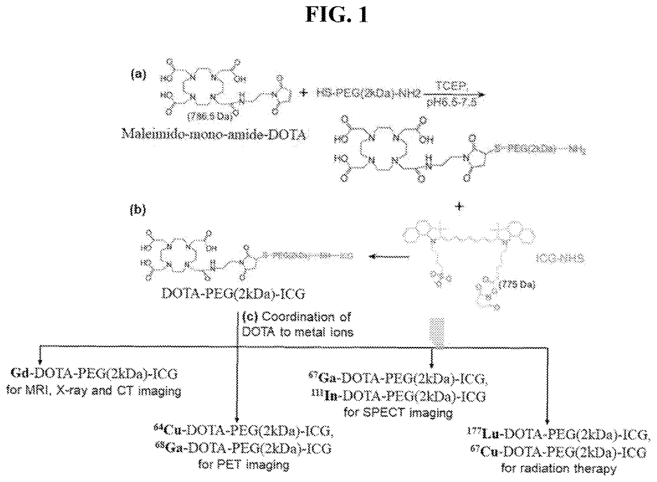

400 μL, 10 mM PEG molecule in ultrapure water was added into 400 μL, 400 μM ICG-NHS in DMSO and the mixture was vortexed for 3 h. Then ICG-PEG conjugates were purified with sephadex column from unconjugated ICG and PEG molecule with mobile phase of ultrapure water. The different mobilities of ICG, ICG-PEG22, ICG-PEG45 and ICG-PEG220 in sephadex column and agarose gel both proved the successful synthesis of ICG-PEG conjugates. The IRDye800CW-PEG45 is synthesized in a similar way with ICG-PEG45 and detailed procedures were reported previously. As shown in FIG. 1, ICG-PEG45 labeled with Gd-DOTA can be used as imaging agents for magnetic resonance imaging (MRI), X-ray and computed tomography (CT) imaging. ICG-PEG45 labeled with ⁶⁴Cu-DOTA or ⁶⁸Ga-DOTA can be used as imaging agents for positron emission tomography (PET). ICG-PEG45 labeled with ⁶⁷Ga-DOTA or ¹¹¹In-DOTA can be used as imaging agents for single-photon emission computerized tomography (SPECT). ICG-PEG45 labeled with ¹⁷⁷Lu-DOTA or ⁶⁷Cu-DOTA can be used as therapeutic radiopharmaceuticals for cancer treatment.

DOTA can be replaced by other chelating ligands that can form complexes with metal ions, such as NOTA (1,4,7-Triazacyclononane-1,4,7-triacetic acid), TETA (1,4,8,11-Tetraazacyclotetradecane-1,4,8, 11-tetraacetic acid), DTPA (diethylenetriaminepentaacetic acid), DO3A-EOB (1,4,7,10-Tetraazacyclododecane-1,4,7-trisacetic Acid (DO3A)-Ethoxybenzyl (EOB)), DOTP (1,4,7,10-Tetraazacyclododecane-1,4,7,10-tetra(methylene phosphonic) acid), Cyclen (1,4,7,10-Tetraazacyclododecane), Cyclam (1,4,8,11-Tetraazacyclotetradecane), CB-TE2A (1,4,8,11-Tetraazabicyclo[6.6.2]hexadecane-4,11-diacetic acid), DOTMA (1R, 4R, 7R, 10R)-α′α″α′″-Tetramethyl-1,4,7,10-tetraazacyclododecane-1,4,7,10-tetraacetic acid) tetrasodium salt, DFO (deferoxamine, a chelator for ⁸⁹Zr).

### Example 2. ICG-PEG45 Serves as an Active Targeting Ligand for Efficiently Delivering Other Imaging Agents and Therapeutic Drugs to Renal Cell Carcinoma (RCC)

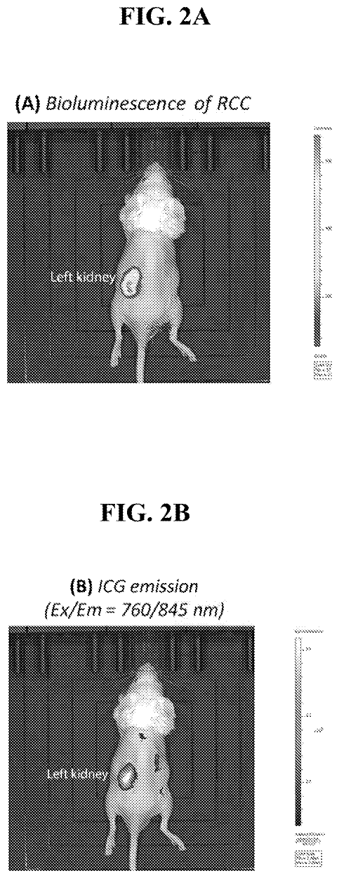

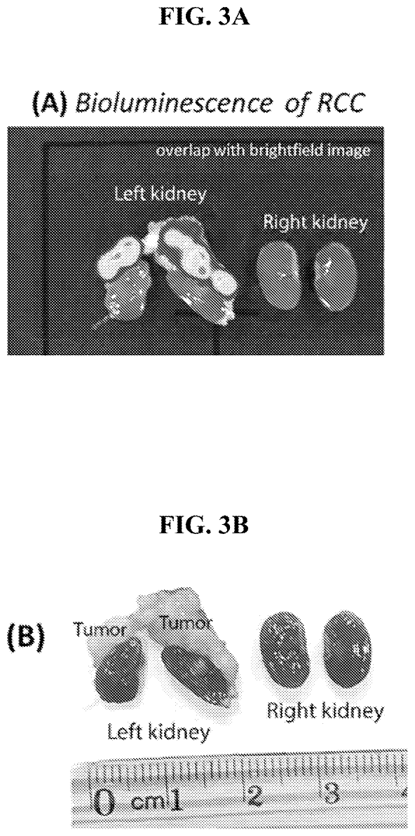

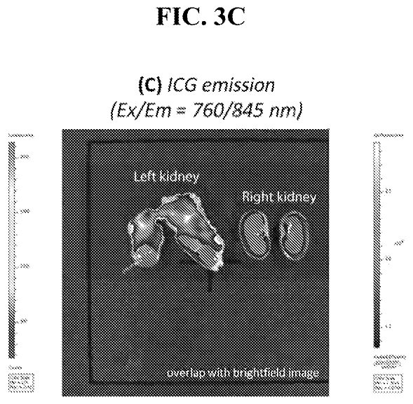

As an example, ICG-PEG45 was conjugated to 1,4,7,10-Tetraazacyclododecane-1,4,7,10-tetraacetic acid (DOTA). DOTA is a clinically used chelating agent that can form complexes with gadolinium for application as MRI contrast agent or form complexes with radioisotopes such as 64Cu and 68Ga for positron emission tomography (PET). PET has become as one of the most important imaging modalities in staging, detecting recurrence and metastasis, and monitoring treatment efficacy in most cancers. However, RCC cannot be accurately diagnosed with PET after injection of [18F]fluorodeoxyglucose (FDG), the most commonly used PET agent, mainly because physiological excretion of FDG from the kidneys reduces the contrast between malignant and normal kidney tissues. To demonstrate the RCC targeting of ICG-PEG45-DOTA, an orthotopic RCC xenograft mouse model was established. The papillary RCC cell line (ACHN) transfected with luciferase-expression vector was surgically implanted into subcapsular space of the left kidney of mice and the right kidney was kept normal for renal function. When the RCC tumor developed to a size that can be reliably detected in the left kidney by bioluminescent imaging, ICG-PEG45-DOTA was intravenously injected into the mice and then conducted noninvasive in vivo fluorescence imaging at 24 h post injection of ICG-PEG45-DOTA (200 μL, 40 μM). As shown in FIG. 2A, strong bioluminescence signal was detected on the left kidney indicated the growth of RCC. At 24 h post injection of ICG-PEG45-DOTA, near-infrared fluorescence image of the same mouse suggests that ICG-PE45-DOTA can specifically accumulate in the left kidney with RCC but can be cleared from the normal right kidney (FIG. 2B). Ex vivo imaging of these two kidneys (FIGS. 3A-3C) further confirmed that the prolonged retention of ICG-PEG45-DOTA in the left kidney specifically occurred in tumor regions. The malignant kidney tissues can be validated by bioluminescence imaging and the tumor was white (FIG. 3A and FIG. 3B). Interestingly, the fluorescence intensity of ICG-PEG45-DOTA in the malignant tissues was higher than that in normal kidney tissues in both left and right kidneys (FIG. 3C). These results clearly indicate that ICG-PEG45 can be used as an active targeting ligand to efficiently deliver other imaging agents and therapeutic drugs to RCC once ICG-PEG45 is conjugated to the agents.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A conjugate, comprising:
a small molecule that binds an influx or efflux transporter; and
an antifouling nanoparticle, wherein
the small molecule is attached to the antifouling nanoparticle through a covalent bond or a metal-ligand bond, and
when the small molecule is indocyanine green (ICG), the antifouling nanoparticle is not polyethylene glycol (PEG), and vice versa.

2. The conjugate of claim 1, wherein the conjugate can be further conjugated to an imaging agent, biochemical activatable agent, or therapeutic agent.

3. The conjugate of claim 2, wherein the imaging agent is a metal-ligand complex or an iodine-containing compound.

4. The conjugate of claim 3, wherein the imaging agent is a metal-ligand complex.

5. The conjugate of claim 4, wherein the ligand comprises DOTA (1,4,7,10-Tetraazacyclododecane-1,4,7,10-tetraacetic acid), NOTA (1,4,7-triazacyclononane-1,4,7-triacetic acid), TETA (1,4,8,11-Tetraazacyclotetradecane-1,4,8, 11-tetraacetic acid), DTPA (diethylenetriaminepentaacetic acid), DO3A-EOB (1,4,7,10-tetraazacyclododecane-1,4,7-trisacetic Acid (DO3A)-Ethoxybenzyl (EOB)), DOTP (1,4,7,10-tetraazacyclododecane-1,4,7,10-tetra(methylene phosphonic) acid), Cyclen (1,4,7,10-Tetraazacyclododecane), Cyclam (1,4,8,11-Tetraazacyclotetradecane), CB-TE2A (1,4,8,11-tetraazabicyclo[6.6.2]hexadecane-4,11-diacetic acid), DOTMA ((1R, 4R, 7R, 10R)-α′α″α′″-tetramethyl-1,4,7,10-tetraazacyclododecane-1,4,7,10-tetraacetic acid) tetrasodium salt), or DFO (deferoxamine).

6. The conjugate of claim 4, wherein the metal comprises Gd, ⁶⁴Cu, ⁶⁷Cu, ⁶⁸Ga, ⁶⁷Ga, ¹¹¹In, ⁸⁹Zr, or ¹⁷⁷Lu.

7. The conjugate of claim 3, wherein the imaging agent is an iodine-containing compound.

8. The conjugate of claim 7, wherein the iodine-containing compound is selected from an iodobenzene, a diiodobenzene, and a triiodobenzene, or derivatives thereof.

9. The conjugate of claim 8, wherein the iodine-containing compound is selected from diatrizoate, metrizoate, iohexol, iopamidol, iopromide, ioxilan, iodixanol, iobitridol, ioversol, iothalamate, and ioxaglate.

10. The conjugate of claim 2, wherein the imaging agent is a gadolinium (Gd) complex, a dysprosium (Dy) complex, or a tantalum oxide-based contrast agent.

11. The conjugate of claim 10, wherein the gadolinium (Gd) complex is selected from gadoversetamide, gadopentetate dimeglumine, gadoterate meglumine, gadoxetate disodium, gadoteridol, and gadobutrol.

12. The conjugate of claim 10, wherein the dysprosium (Dy) complex is selected from Dy-EOB-DTPA [(4S)-4-(4-ethoxybenzyl)-3,6,9-tris-(carboxylatomethyl)-3,6,9-triazaundecanedioic acid, dysprosium complex, and disodium salt.

13. The conjugate of claim 2, wherein the imaging agent is for positron emission tomography (PET) and is a compound labeled by a radioisotope.

14. The conjugate of claim 13, wherein the radioisotope is ¹¹C, ¹⁸F, ⁶⁴Cu, ⁶⁸Ga, or ⁸⁹Zr.

15. The conjugate of claim 2, wherein the imaging agent is for single-photon emission computerized tomography (SPECT) and is a compound labeled by a radioisotope.

16. The conjugate of claim 15, wherein the radioisotope is ^{99m}Tc, ¹²³I, ¹²⁵I, ¹³¹I, ⁶⁷Ga, or ¹¹¹In.

17. The conjugate of claim 2, wherein the imaging agent is a gadolinium (Gd)-based MRI contrast agent, manganese (Mn)-based MRI contrast agent, or an iron oxide nanoparticle for magnetic resonance imaging (MRI).

18. The conjugate of claim 17, wherein the gadolinium (Gd)-based MRI contrast agent is selected from gadoversetamide, gadopentetate dimeglumine, gadoterate meglumine, gadoxetate disodium, gadoteridol, and gadobutrol.

19. The conjugate of claim 17, wherein the manganese (Mn)-based MRI contrast agent is selected from the semistable chelate manganese dipyridoxyl diphosphate (MnDPDP), Mn chelated with ethylenediaminetetraacetic acid (Mn-EDTA), and Mn chelated with diethylene triamine pentaacetic acid (Mn-DTPA).

20. The conjugate of claim 2, wherein the therapeutic agent comprises an antioxidant used for preventing or treating oxidative stress-related diseases.

21. The conjugate of claim 20, wherein the antioxidant is N-acetylcysteine, alfa-lipoic acid, bilirubin or dihydrolipoic acid.

22. The conjugate of claim 2, wherein the therapeutic agent comprises a small molecule drug for chemotherapy.

23. The conjugate of claim 22, wherein the small molecule drug for chemotherapy is cisplatin, carboplatin, doxorubicin, epirubicin, paclitaxel, docetaxel, methotrexate, capecitabine, sorafenib, sirolimus, temsirolimus, or everolimus.

24. The conjugate of claim 2, wherein the therapeutic agent comprises a compound containing a therapeutic radioisotope.

25. The conjugate of claim 24, wherein the therapeutic radioisotope is ¹⁷⁷Lu, ⁶⁷Cu, or ¹⁹⁸Au.

26. The conjugate of claim 2, wherein the therapeutic agent comprises a peptide, an antibody or a fragment thereof, an immunoagent, a RNA, a mRNA, or a DNA.

27. The conjugate of any one of claims 1-26, wherein the small molecule that binds an influx or efflux transporter is a dye.

28. The conjugate of any one of claims 1-27, wherein the influx or efflux transporter is P-glycoprotein (P-gp), organic anion transporters (OATs), organic anion transporting polypeptides (OATPs), organic cation transporters (OCTs), multidrug-resistant proteins (MRPs), adenosine triphosphate (ATP) binding cassette (ABC)-type MDR transporters, or breast cancer resistance protein (BCRP).

29. The conjugate of claim 27, wherein the dye is a near-infrared cyanine dye selected from indocyanine green (ICG), IR-780, IR-783, MHI-148, and DZ-1.

30. The conjugate of claim 27, wherein the dye is ICG.

31. The conjugate of claim 27, wherein the dye is a rhodamine dye selected from near-infrared dye Rhodamine 800, Rhodamine-123, Tetramethylrhodamine (TMR), Tetramethylrhodamine methyl ester (TMRM), and Rhodamine 6G.

32. The conjugate of claim 27, wherein the dye is a cationic carbocyanine dye selected from DiOC₆(3) (3,3′-dihexyloxacarbocyanine Iodide), DiOC₂(3) (3,3′-diethyloxacarbocyanine, iodide), and DisC₃(5) (3,3′-dipropylthiadicarbocyanine iodide).

33. The conjugate of claim 27, wherein the dye is fluorescein.

34. The conjugate of claim 27, wherein the dye is a DNA stain selected from Hoechst 33342, DyeCycle Violet (DCV), Ethidium bromide, and SYBR Green.

35. The conjugate of claim 27, wherein the dye is bilirubin ditaurate.

36. The conjugate of any one of claims 1-35, wherein the antifouling nanoparticle is an antifouling macromolecule.

37. The conjugate of claim 36, wherein the antifouling macromolecule is a biopolymer.

38. The conjugate of claim 37, wherein the biopolymer comprises a carbohydrate, a lipid, a protein, a peptide, or a nucleic acid.

39. The conjugate of claim 36, wherein the antifouling macromolecule is a synthetic polymer.

40. The conjugate of claim 36, wherein the antifouling macromolecule is a hydrophilic polymer selected from polyether, polysaccharide, polyacrylamide, polyacrylate, polyamide, polypeptoids, β-peptoid, poly(β-peptoid)s, and polyalkyloxazoline.

41. The conjugate of claim 40, wherein the hydrophilic polymer is selected from ethylene glycol-based polymers, polyethylene glycol (PEG), poly(2-hydroxyethyl methacrylate) (pHEMA), poly(hydroxypropyl methacrylate) (pHPMA), dextran, and cellulose.

42. The conjugate of claim 41, wherein the hydrophilic polymer is PEG.

43. The conjugate of claim 36, wherein the antifouling macromolecule is a zwitterionic polymer selected from polybetaine (with positive and negative charges in series on the same side chain), mixed cationic-anionic pairs (with positive and negative charges on two different side chains), and Bingdi cationic-anionic pairs (with positive and negative charges in parallel on the same side chain, such as cysteine).

44. The conjugate of claim 43, wherein the zwitterionic polymer is polycarboxybetaine, polysulfobetaine, or polyphosphorylcholine.

45. The conjugate of claim 44, wherein the zwitterionic polymer is selected from poly(carboxybetaine methacrylate) (pCBMA), poly(sulfobetaine methacrylate) (pSBMA), or poly(2-methacryloyloxyethyl phosphorylcholine) (pMPC).

46. The conjugate of claim 43, wherein the zwitterionic polymer is poly(serine methacrylate) (pSerMA), poly(lysine methacrylamide) (pLysAA), poly(omithine methacrylamide) (pOmAA), or polyampholyte mixed-charge copolymers composed of positively charged quaternary amine monomers or negatively charged monomers.

47. The conjugate of claim 46, wherein the zwitterionic polymer is a polyampholyte mixed-charge copolymer comprising positively charged quaternary amine monomers or negatively charged monomers.

48. The conjugate of claim 47, wherein the positively charged quaternary amine monomer or negatively charged monomer is [2-(acryloyloxy)ethyl]trimethylammonium chloride or [2-(methacryloyloxy) ethyl]trimethylammonium chloride, 2-carboxy ethyl acrylate, or 3-sulfopropyl methacrylate potassium salt.

49. The conjugate of any one of claims 1-48, wherein the antifouling macromolecule has an average molecular weight of about 1,000 to about 100,000 Dalton.

50. The conjugate of any one of claims 1-35, wherein the antifouling nanoparticle is an inorganic nanoparticle.

51. The conjugate of claim 50, wherein the inorganic nanoparticle comprises gold, silver, copper, platinum, palladium, silica, carbon, silicon, iron oxide, FeS, CdSe, CdS, CuS, or a combination thereof.

52. The conjugate of any one of claims 1-51, wherein the antifouling nanoparticle has an average diameter of about 0.5 nm to about 12 nm.

53. The conjugate of any one of claims 1-52, wherein the conjugate has a one-hour or two-hour renal clearance efficiency in the range of 0.5 to 100 percent of injected dose (% ID).

54. A conjugate comprising indocyanine green (ICG), polyethylene glycol (PEG), and a secondary moiety, wherein ICG and the secondary moiety are each independently conjugated to PEG.

55. The conjugate of claim 54, wherein the secondary moiety is an imaging agent or therapeutic agent.

56. A method of diagnosing a disease or condition associated with abnormal expression of an influx or efflux transporter in a subject, comprising:
i) administering to the subject a conjugate of any one of claims 1-55;
ii) determining a concentration of the conjugate in a biological sample obtained from the subject;
iii) comparing the concentration of the conjugate with a reference level; and
iv) determining that the subject has the disease or condition if the concentration of the conjugate is significantly greater or lower than the reference level.

57. A method of diagnosing a disease or condition associated with abnormal expression of an influx or efflux transporter in a subject, comprising:
i) administering to the subject a conjugate of any one of claims 1-55;
ii) measuring an intensity of a signal from the conjugate in a tissue of the subject;
iii) comparing the intensity with a reference level; and
iv) determining that the subject has the disease or condition if the intensity is significantly greater or lower than the reference level.

58. A method of monitoring kidney secretion function of a subject, comprising:
i) administering to the subject a conjugate of any one of claims 1-55;
ii) determining a first concentration of the conjugate in a first biological sample obtained from the subject at a first time point;
iii) determining a second concentration of the conjugate in a second biological sample obtained from the subject at a second time point, wherein the second time point is after the first time point;
iv) determining renal clearance kinetics based on the first concentration and the second concentration; and
v) optionally comparing the renal clearance kinetics with a reference level.

59. A method of monitoring kidney secretion function of a subject, comprising:
i) administering to the subject a conjugate of any one of claims 1-55;
ii) measuring, at a first time point, a first intensity of a signal from the conjugate in a tissue of the subject; and
iii) measuring, at a second time point, a second intensity of a signal from the conjugate in the tissue of the subject.

60. A method of treating a disease or condition associated with abnormal expression of an influx or efflux transporter in a subject in need thereof, comprising administering to the subject a conjugate of any one of claims 1-55.

61. A method of detecting a liver disease in a subject, comprising:
i) administering to the subject a conjugate of any one of claims 1-55;
ii) determining a concentration of the conjugate in a biological sample obtained from the subject;
iii) comparing the concentration of the conjugate with a reference level; and
iv) determining that the subject has the liver disease when the concentration of the conjugate is significantly greater or lower than the reference level.

62. A method of measuring an expression level of an influx or efflux transporter in a subject, comprising:
i) administering to the subject a conjugate of any one of claims 1-55;
ii) determining a concentration of the conjugate in a biological sample obtained from the subject; and
iii) determining the expression level of the influx or efflux transporter based on the concentration of the conjugate.

63. A method of measuring an expression level of an influx or efflux transporter in a subject, comprising:
i) administering to the subject a conjugate of any one of claims 1-55;
ii) measuring an intensity of a signal from the conjugate in a tissue of the subject; and
iii) determining the expression level of the influx or efflux transporter based on the intensity.

64. The method of any one of claims 56-63, wherein the subject has upregulated or downregulated expression of P-glycoprotein (P-gP), multidrug-resistant protein 2 (MRP2), MRP4, an organic cation transporter (OCT), an organic anion transporter (OAT), an organic anion-transporting polypeptide (OATP), breast cancer resistance protein (BCRP), or organic anion transporter 4 (OAT4), equilibrative nucleoside transporter 1 (ENT1), ENT2, organic solute transporter α (OSTα), or OSTβ.

65. The method of any one of claims 56-64, wherein the disease or condition is renal tubular secretion dysfunction or renal tubular injury.

66. The method of claim 65, wherein the renal tubular secretion dysfunction or renal tubular injury is proximal renal tubular secretion dysfunction or proximal renal tubular injury.

67. The method of claim 65 or 66, wherein the renal tubular secretion dysfunction or renal tubular injury is associated with a kidney disease or condition selected from acute kidney injury, chronic kidney injury, kidney cancer, lupus nephritis, diabetes-induced kidney injury, polycystic kidney disease, sepsis, kidney inflammation, kidney transplant rejection, and kidney dysfunction or kidney injury caused by diseases in other tissues and organs such as cancer and liver diseases.

68. The method of any one of claims 56-64, wherein the disease or condition is kidney cancer, breast cancer, liver cancer, ovarian cancer, bladder cancer, prostate cancer, lung cancer, pancreatic cancer, bone cancer, or colon cancer.

69. The method of claim 68, wherein the kidney cancer is renal cell carcinoma or renal oncocytoma.

70. The method of claim 69, wherein the kidney cancer is renal cell carcinoma.

71. The method of claim 70, wherein the renal cell carcinoma is clear cell renal cell carcinoma (ccRCC), or papillary RCC (pRCC) or its metastases.

72. The method of claim 68, wherein the disease or condition is breast cancer, and wherein the breast cancer is triple negative breast cancer.

73. The method of any one of claims 56-72, wherein the biological sample is a blood or urine sample.

74. The method of any one of claims 56-72, wherein the biological sample is a urine sample.

75. The method of any one of claims 56-74, wherein the conjugate is administered intravenously, intraperitoneally, subcutaneously, or intraarterially.
